(19) United States
(12) Patent Application Publication
(10) Pub. No.: US 2018/0318344 A1
(43) Pub. Date: Nov. 8, 2018

(54) ZINC OR MANGANESE COMPOUNDS AS THERAPEUTIC ADJUNCTS FOR CARTILAGE REGENERATION AND REPAIR

(71) Applicant: Rutgers, The State University of New Jersey, New Brunswick, NJ (US)

(72) Inventors: Sheldon S. Lin, Chatham, NJ (US); David N. Paglia, Framingham, MA (US); James P. O'Connor, Suffern, NY (US); Aaron Wey, East Brunswick, NJ (US); Joseph Benevenia, Montclair, NJ (US); Virak Tan, Short Hill, NJ (US)

(21) Appl. No.: 15/943,369

(22) Filed: Apr. 2, 2018

Related U.S. Application Data
(63) Continuation-in-part of application No. 14/130,830, filed on Jul. 2, 2014, now Pat. No. 9,931,348.
Application No. 14/130,830 is a continuation-in-part of application No. 14/359,827, filed on May 21, 2014.
Application No. 14/359,827 is a continuation-in-part of application No. PCT/US11/64240, filed on Dec. 9, 2011.
(60) Provisional application No. 61/504,777, filed on Jul. 6, 2011.
(60) Provisional application No. 61/718,646, filed on Oct. 25, 2012.
(60) Provisional application No. 61/564,822, filed on Nov. 29, 2011.
(60) Provisional application No. 61/454,061, filed on Mar. 18, 2011.
(60) Provisional application No. 61/428,342, filed on Dec. 30, 2010.
(60) Provisional application No. 61/421,921, filed on Dec. 10, 2010.

(51) Int. Cl. A61K 33/30; A61K 33/32; A61P 19/04; A61K 45/06

(52) U.S. Cl. CPC A61K 33/30; A61K 45/06; A61P 19/04; A61K 33/32

(57) ABSTRACT

A method for repairing an injury of cartilage in a patient by local administration of a zinc or manganese agent or use of an implantable device for delivery of an a zinc or manganese agent. Implantable devices containing a zinc or manganese agent and methods of making these implantable devices are also disclosed.

### CROSS-REFERENCE TO RELATED APPLICATIONS

This application is a Continuation-In-Part to U.S. application Ser. No. 14/130,830, filed Jan. 3, 2014, which is a U.S. National Stage Application of International Application No. PCT/US2012/045771, filed Jul. 6, 2012, which claims priority to U.S. Provisional Application Ser. No. 61/504,777, filed Jul. 6, 2011, all of which are hereby incorporated by reference in their entirety. This application is also a Continuation-In-Part to U.S. application Ser. No. 14/359,827, filed May 21, 2014, which is a U.S. National Stage Application of International Application No. PCT/US2012/067087, filed Nov. 29, 2012, which claims priority to U.S. Provisional Application Ser. No. 61/718,646, filed Oct. 25, 2012 and U.S. Provisional Patent Application Ser. No. 61/564,822, filed Nov. 29, 2011. PCT/US2012/067087 is a Continuation-In-Part to International Application No. PCT/US2011/064240, filed on Dec. 9, 2011, which claims priority to U.S. Provisional Patent Application Ser. No. 61/421,921, filed on Dec. 10, 2010, U.S. Provisional Patent Application Ser. No. 61/428,342, filed on Dec. 30, 2010, and U.S. Provisional Patent Application Ser. No. 61/454,061, filed on Mar. 18, 2011, all of which are hereby incorporated by reference in their entireties.

### FIELD OF THE INVENTION

The present invention relates to compositions or devices comprising zinc and manganese compounds as therapeutic adjuncts for cartilage regeneration and repair.

### BACKGROUND OF THE INVENTION

Articular cartilage has little capacity to repair itself or regenerate intrinsically. Therefore, cartilage defects repair by forming scar tissue (or fibrocartilage) from the subchondral bone. This scar tissue is deficient in type II collagen and has “abnormal” proteoglycans (which have inferior biomechanical characteristics) and lower load bearing capacity, and its formation will often result in short term recovery only. This later surface deterioration may progress to give chronic pain and poor function and may in some cases lead to early onset osteoarthritis.

A regional database study of over 30,000 patients found that 63% of knees that undergo arthroscopy are found to have disease in the articular cartilage, and articular chondral lesions are suspected to be the cause of as many as 10% of all knee hemarthroses. Trauma is the most common etiology, but other conditions, such as osteochondritis dissecans and chondromalacia patellae (abnormal softening of the patellar articular cartilage), are also accepted as causes of symptomatic painful articular lesions. Isolated articular cartilage injuries secondary to trauma are rare; more often articular cartilage injuries are seen with other traumatic injuries to the knee, such as ligamentous or meniscal damage.

Osteochondral lesions (and osteochodritis dessicans) are common in adolescents. A recent magnetic resonance imaging study found that after acute trauma the most common injuries to the immature knee were chondral in nature. Traumatic forces are transmitted through the subchondral bone beneath the cartilage, resulting in an osteochondral fracture. Treatment of larger and symptomatic lesions is often surgical. Ideally the aim of surgery is to provide an environment that allows whatever repair tissue is produced (preferably hyaline cartilage) to be integrated with native healthy tissue to provide long term durability and a “normal” knee joint.

In recent years, the potential use of zinc and manganese as an alternative or adjunct treatment for diabetes has been examined. However, the effects of zinc and manganese compounds on cartilage healing and regeneration are unknown. In particular, no evaluation of zinc or manganese therapy on cartilage regeneration, in particular, repairing of cartilage injuries, has been performed, and in vivo data on cartilage regeneration or repair in the presence of zinc or manganese are still unavailable.

### SUMMARY OF THE INVENTION

The present invention provides a novel method for accelerating cartilage healing or repair using zinc or manganese compounds. The present invention thus obviates the need for developing specialized methods to deliver growth factors and thereby reduces costs associated with therapy, eliminates specialized storage and enhances ease of use.

In one aspect the present invention provides a method for repairing an injury of cartilage in a patient in need thereof by locally administering a therapeutically effective amount of a zinc or manganese compound to the patient.

In another aspect the present invention provides a method for repairing an injury of cartilage in a patient in need thereof by treating the patient with an implantable device having a composite surface coating containing a zinc or manganese compound.

In another aspect the present invention provides an implantable device for implant in a cartilage to treat an injury of the cartilage, containing a zinc or manganese compound.

In another aspect the present invention provides use of a zinc or manganese compound or composition thereof for manufacture of a medicament or device for repairing a cartilage injury.

The therapeutic adjunct of the present invention may find application in, e.g., traumatic cartilaginous injuries, osteochondral lesions, osteochondral fracture, osteochondritis dissecans, chondromalacia, and avascular necrosis. Application of the present invention as therapeutic cartilaginous adjunct will also enhance the currently utilized surgical techniques.

The present invention may find wide application in veterinary medicines to treat a variety of factures in a mammalian animal, including but not limited to, horses, dogs, cats, or any other domestic or wild mammalian animals. A particular useful application may be found, for example, in treating an injured race horse. Other aspects and embodiments of the present invention will be further illustrated in the following description and examples.

### BRIEF DESCRIPTION OF THE DRAWINGS

FIG. 1 depicts post-operative X-rays. Representative x-rays taken immediately post-operative: (A) Einhorn model, (B) model used in this work. (Note in (B) the Kirschner wire is going through the trochanter, which helps to stabilize the fracture site and prevent the migration of the Kirschner wire.)

FIG. 2 depicts Mechanical Testing Setup: Intact femur before embedded in/4 inch square nut with Field's Metal, where (A) ZINC 10 (3.0 mg/kg ZnCl2) and (B) ZINC 8 (1.0 mg/kg ZnCl₂) represent two sets of Zinc treated femurs harvested 4 weeks post-surgery, showing spiral fracture indicative of healing, compared to (C) ZINC 3 (control) showing non-spiral fracture indicative of non-union (Left: Intact Femur, Right: Fractured Femur).

FIG. 3 illustrates 4-week radiographs (AP and Medial-Lateral views) of representative samples of fracture femur bones treated with local ZnCl₂ (1.0 and 3.0 mg/Kg) in comparison with saline control.

FIG. 4 illustrates histomorphometry of ZnCl₂ treated fractures in comparison with saline control.

FIG. 5 illustrates 4-week radiographs (AP and Medial-Lateral views) of representative sample for each group of fractured femur bones treated with 1.0 mg/Kg ZnCl₂+CaSO₄ carrier in comparison with CaSO₄ control.

FIG. 6 illustrates comparison of use of ZnCl₂ with the existing therapy (BMP2): (1) a single intramedullary dose (1 mg/kg) of ZnCl₂ with the calcium sulfate (CaSO₄) vehicle (purple); (2) a single intramedullary dose (3 mg/kg) of ZnCl₂ without a vehicle (green); (3) BMP-2 study used a single percutaneous dose of BMP-2 (80 μg) with buffer vehicle (red); and (4) Exogen study used daily exposure periods of ultrasound treatment (20 min/day). The average value (duration of 25 days) is shown in blue.

FIG. 7 illustrates 4-week post-fracture radiographs of local manganese chloride (MnCl₂) treatment group vs. saline control.

### DETAILED DESCRIPTION OF THE INVENTION

The present invention incorporates the discovery that zinc- or manganese-containing agents play a critical role in cartilage repairing and regeneration. In one aspect the present invention provides a method for repairing an injury of a cartilage in a patient in need thereof, by locally administering a therapeutically effective amount of a zinc or manganese compound to a patient.

In one embodiment of this aspect, the zinc compounds suitable for the present invention include inorganic zinc compounds, such as mineral acid zinc salts. Examples of inorganic zinc compounds include, but are not limited to, zinc chloride, zinc sulfate, zinc phosphate, zinc carbonate, and zinc nitrate, or combinations thereof.

The zinc compound may also be zinc salts of organic acids. Examples of organic acid zinc salts include, but are not limited to, zinc acetate, zinc formate, zinc propionate, zinc gluconate, bis(maltolato)zinc, zinc acexamate, zinc aspartate, bis(maltolato)zinc(II) [Zn(ma)2], bis(2-hydroxypyridine-N-oxido)zinc(II) [Zn(hpo)2], bis(allixinato)Zn(II) [Zn(alx)2], bis(6-methylpicolinato)Zn(II) [Zn(6mpa)2], bis(aspirinato)zinc(II), bis(pyrrole-2-carboxylato)zinc [Zn(pc)2], bis(alpha-furonic acidato)zinc [Zn(fa)2], bis(thiophene-2-carboxylato)zinc [Zn(tc)2], bis(thiophene-2-acetato)zinc [Zn(ta)2], (N-acetyl-L-cysteinato)Zn(II) [Zn(nac)], zinc(II)/poly(γ-glutamic acid) [Zn(γ-pga)], bis(pyrrolidine-N-dithiocarbamate)zinc(II) [Zn(pdc)₂], zinc(II) L-lactate [Zn(lac)₂], zinc(II) D-(2)-quinic acid [Zn(qui)₂], bis(1,6-dimethyl-3-hydroxy-5-methoxy-2-pentyl-1,4-dihydropyridine-4-thionato)zinc(II) [Zn(tanm)2], β-alanyl-L-histidinato zinc(II) (AHZ), or the like, or combinations thereof. In another embodiment, the organic acid of zinc salt is a naturally occurring fatty acid.

In one embodiment of this aspect, the manganese compounds suitable for the present invention include, but are not limited to, manganese chloride (MnCl₂), 3-O-methyl-D-chiro-inositol+manganese chloride (MnCl₂), D-chiro-inositol+manganese chloride (MnCl₂), manganese sulfate [MnSO4], inositol glycan pseudo-disaccharide Mn(2+) chelate containing D-chiro-inositol 2a (as pinitol) and galactosamine, oral manganese, manganese oxides, e.g., MnO₂, MnOAl₂O₃, and Mn₃O₄.

In another embodiment of this aspect, the cartilage injury is selected from traumatic cartilaginous injuries, osteochondral lesions, osteochondral fracture, osteochondritis dissecans, chondromalacia, avascular necrosis, chemical induced cartilage damage (e.g., steroid injection), and genetic cartilage deficiency, or the like.

In another embodiment of this aspect, the cartilage is an articular cartilage.

In another embodiment of this aspect, the method is used in conjunction with arthroscopic debridement, marrow stimulating techniques, autologous chondrocyte transfers, and autologous chondrocyte implantation, and allografts.

In another embodiment of this aspect, the method is used in conjunction with administration of a cytototoxic agent, cytokine or growth inhibitory agent.

In another embodiment of the present invention, the method is used in conjunction with an allograft/autograft or orthopedic biocomposite.

In another embodiment of this aspect, the patient is a mammalian animal.

In another embodiment of this aspect, the patient is a human.

In another embodiment of this aspect, the patient is a non-diabetic human.

In another embodiment of this aspect, the patient is a horse or dog.

In another preferred embodiment of this aspect, the present invention is particularly suitable for, but is not limited to, repairing cartilage tissue damages that are caused by long term or sudden trauma or injury.

In another aspect the present invention provides a method for repairing an injury of a cartilage in a patient in need thereof comprising treating said patient with an implantable device comprising a zinc or manganese compound. The implantable device can be a delivery system of a composition containing the zinc or manganese compound, a zinc- or manganese-coated orthopedic implant, or an article that also provides support to an injured or damaged joint.

Zinc compounds suitable for the present invention include inorganic zinc compounds, such as mineral acid zinc salts. Examples of inorganic zinc compounds include, but are not limited to, zinc chloride, zinc sulfate, zinc phosphate, zinc carbonate, and zinc nitrate, or combinations thereof.

The zinc compound may also be zinc salts of organic acids. Examples of organic acid zinc salts include, but are not limited to, zinc acetate, zinc formate, zinc propionate, zinc gluconate, bis(maltolato)zinc, zinc acexamate, zinc aspartate, bis(maltolato)zinc(II) [Zn(ma)2], bis(2-hydroxypyridine-N-oxido)zinc(II) [Zn(hpo)2], bis(allixinato)Zn(II) [Zn(alx)2], bis(6-methylpicolinato)Zn(II) [Zn(6mpa)2], bis(aspirinato)zinc(II), bis(pyrrole-2-carboxylato)zinc [Zn(pc)2], bis(alpha-furonic acidato)zinc [Zn(fa)2], bis(thiophene-2-carboxylato)zinc [Zn(tc)2], bis(thiophene-2-acetato)zinc [Zn(ta)2], (N-acetyl-L-cysteinato)Zn(II) [Zn(nac)], zinc(II)/poly(γ-glutamic acid) [Zn(γ-pga)], bis(pyrrolidine-N-dithiocarbamate)zinc(II) [Zn(pdc)₂], zinc(II) L-lactate [Zn(lac)₂], zinc(II) D-(2)-quinic acid [Zn(qui)₂], bis(1,6-dimethyl-3-hydroxy-5-methoxy-2-pentyl-1,4-dihydropyridine-4-thionato)zinc(II) [Zn(tanm)2], β-alanyl-L-histidinato zinc(II) (AHZ), or the like, or combinations thereof. In another embodiment, the organic acid of zinc salt is a naturally occurring fatty acid.

Manganese compounds suitable for the present invention include, but are not limited to, manganese chloride (MnCl₂), 3-O-methyl-D-chiro-inositol+manganese chloride (MnCl₂), D-chiro-inositol+manganese chloride (MnCl₂), manganese sulfate [MnSO4], inositol glycan pseudo-disaccharide Mn(2+) chelate containing D-chiro-inositol 2a (as pinitol) and galactosamine, oral manganese, manganese oxides, e.g., MnO₂, MnOAl₂O₃, and Mn₃O₄.

In another embodiment of this aspect, the cartilage injury is selected from traumatic cartilaginous injuries, osteochondral lesions, osteochondral fracture, osteochondritis dissecans, chondromalacia, avascular necrosis, chemical induced cartilage damage (e.g., steroid injection), and genetic cartilage deficiency, or the like.

In another embodiment of this aspect, the cartilage injury is that of an articular cartilage.

In another embodiment of this aspect, the method is used in conjunction with arthroscopic debridement, marrow stimulating techniques, autologous chondrocyte transfers, and autologous chondrocyte implantation, and allografts.

In another embodiment of this aspect, the method is used in conjunction with administration of a cytototoxic agent, cytokine or growth inhibitory agent.

In another embodiment of this aspect, the method is used in conjunction with an allograft/autograft or orthopedic biocomposite.

In another embodiment of this aspect, the patient is a mammalian animal.

In another embodiment of this aspect, the patient is a human.

In another embodiment of this aspect, the patient is a non-diabetic human.

In another embodiment of this aspect, the patient is a horse or dog.

In another preferred embodiment of this aspect, the present invention is particularly suitable for, but is not limited to, repairing cartilage tissue damages that are caused by long term or sudden trauma, injury and/or diseases.

In another aspect the present invention provides an implantable device for implant in cartilage tissue to treat an injury of the cartilage containing a zinc or manganese compound. In one embodiment of this aspect, the zinc compound is an inorganic zinc compounds, such as mineral acid zinc salts. Examples of inorganic zinc compounds include, but are not limited to, zinc chloride, zinc sulfate, zinc phosphate, zinc carbonate, and zinc nitrate, or combinations thereof.

The zinc compound may also be zinc salts of organic acids. Examples of organic acid zinc salts include, but are not limited to, zinc acetate, zinc formate, zinc propionate, zinc gluconate, bis(maltolato)zinc, zinc acexamate, zinc aspartate, bis(maltolato)zinc(II) [Zn(ma)2], bis(2-hydroxypyridine-N-oxido)zinc(II) [Zn(hpo)2], bis(allixinato)Zn(II) [Zn(alx)2], bis(6-methylpicolinato)Zn(II) [Zn(6mpa)2], bis(aspirinato)zinc(II), bis(pyrrole-2-carboxylato)zinc [Zn(pc)2], bis(alpha-furonic acidato)zinc [Zn(fa)2], bis(thiophene-2-carboxylato)zinc [Zn(tc)2], bis(thiophene-2-acetato)zinc [Zn(ta)2], (N-acetyl-L-cysteinato)Zn(II) [Zn(nac)], zinc(II)/poly(γ-glutamic acid) [Zn(γ-pga)], bis(pyrrolidine-N-dithiocarbamate)zinc(II) [Zn(pdc)₂], zinc(II) L-lactate [Zn(lac)₂], zinc(II) D-(2)-quinic acid [Zn(qui)₂], bis(1,6-dimethyl-3-hydroxy-5-methoxy-2-pentyl-1,4-dihydropyridine-4-thionato)zinc(II) [Zn(tanm)2], β-alanyl-L-histidinato zinc(II) (AHZ), or the like, or combinations thereof. In another embodiment, the organic acid of zinc salt is a naturally occurring fatty acid.

In one embodiment of this aspect, the manganese compound may include, but is not limited to, manganese chloride (MnCl₂), 3-O-methyl-D-chiro-inositol+manganese chloride (MnCl₂), D-chiro-inositol+manganese chloride (MnCl₂), manganese sulfate [MnSO4], inositol glycan pseudo-disaccharide Mn(2+) chelate containing D-chiro-inositol 2a (as pinitol) and galactosamine, oral manganese, manganese oxides, e.g., MnO₂, MnOAl₂O₃, and Mn₃O₄.

In another preferred embodiment of this aspect, the device is coated by a composite surface coating containing a zinc or manganese compound. In another preferred embodiment of this aspect, the zinc compound is an inorganic zinc compounds, such as mineral acid zinc salts. Examples of inorganic zinc compounds include, but are not limited to, zinc chloride, zinc sulfate, zinc phosphate, zinc carbonate, and zinc nitrate, or combinations thereof.

The zinc compound may also be zinc salts of organic acids. Examples of organic acid zinc salts include, but are not limited to, zinc acetate, zinc formate, zinc propionate, zinc gluconate, bis(maltolato)zinc, zinc acexamate, zinc aspartate, bis(maltolato)zinc(II) [Zn(ma)2], bis(2-hydroxypyridine-N-oxido)zinc(II) [Zn(hpo)2], bis(allixinato)Zn(II) [Zn(alx)2], bis(6-methylpicolinato)Zn(II) [Zn(6mpa)2], bis(aspirinato)zinc(II), bis(pyrrole-2-carboxylato)zinc [Zn(pc)2], bis(alpha-furonic acidato)zinc [Zn(fa)2], bis(thiophene-2-carboxylato)zinc [Zn(tc)2], bis(thiophene-2-acetato)zinc [Zn(ta)2], (N-acetyl-L-cysteinato)Zn(II) [Zn(nac)], zinc(II)/poly(γ-glutamic acid) [Zn(γ-pga)], bis(pyrrolidine-N-dithiocarbamate)zinc(II) [Zn(pdc)₂], zinc(II) L-lactate [Zn(lac)₂], zinc(II) D-(2)-quinic acid [Zn(qui)₂], bis(1,6-dimethyl-3-hydroxy-5-methoxy-2-pentyl-1,4-dihydropyridine-4-thionato)zinc(II) [Zn(tanm)2], β-alanyl-L-histidinato zinc(II) (AHZ), or the like, or combinations thereof. In another embodiment, the organic acid of zinc salt is a naturally occurring fatty acid.

In one embodiment of this aspect, the manganese compound may include, but is not limited to, manganese chloride (MnCl₂), 3-O-methyl-D-chiro-inositol+manganese chloride (MnCl₂), D-chiro-inositol+manganese chloride (MnCl₂), manganese sulfate [MnSO4], inositol glycan pseudo-disaccharide Mn(2+) chelate containing D-chiro-inositol 2a (as pinitol) and galactosamine, oral manganese, manganese oxides, e.g., MnO₂, MnOAl₂O₃, and Mn₃O₄.

In another embodiment of this aspect, the present invention is particularly suitable for, but is not limited to, repairing cartilage tissue damages that are caused by long term or sudden trauma or injury.

Another aspect of the present invention provides the use of a zinc of manganese compound or composition thereof for the manufacture of a medicament or device for treatment of a cartilage injury, in particular, without limitations, cartilage tissue damages that are caused by long term or sudden trauma or injury.

In a preferred embodiment of this aspect, the zinc compound is an inorganic zinc compounds, such as mineral acid zinc salts. Examples of inorganic zinc compounds include, but are not limited to, zinc chloride, zinc sulfate, zinc phosphate, zinc carbonate, and zinc nitrate, or combinations thereof.

The zinc compound may also be zinc salts of organic acids. Examples of organic acid zinc salts include, but are not limited to, zinc acetate, zinc formate, zinc propionate, zinc gluconate, bis(maltolato)zinc, zinc acexamate, zinc aspartate, bis(maltolato)zinc(II) [Zn(ma)2], bis(2-hydroxypyridine-N-oxido)zinc(II) [Zn(hpo)2], bis(allixinato)Zn(II) [Zn(alx)2], bis(6-methylpicolinato)Zn(II) [Zn(6mpa)2], bis(aspirinato)zinc(II), bis(pyrrole-2-carboxylato)zinc [Zn(pc)2], bis(alpha-furonic acidato)zinc [Zn(fa)2], bis(thiophene-2-carboxylato)zinc [Zn(tc)2], bis(thiophene-2-acetato)zinc [Zn(ta)2], (N-acetyl-L-cysteinato)Zn(II) [Zn(nac)], zinc(II)/poly(γ-glutamic acid) [Zn(γ-pga)], bis(pyrrolidine-N-dithiocarbamate)zinc(II) [Zn(pdc)₂], zinc(II) L-lactate [Zn(lac)₂], zinc(II) D-(2)-quinic acid [Zn(qui)₂], bis(1,6-dimethyl-3-hydroxy-5-methoxy-2-pentyl-1,4-dihydropyridine-4-thionato)zinc(II) [Zn(tanm)2], β-alanyl-L-histidinato zinc(II) (AHZ), or the like, or combinations thereof. In another embodiment, the organic acid of zinc salt is a naturally occurring fatty acid.

In one embodiment of this aspect, the manganese compound may include, but is not limited to, manganese chloride (MnCl₂), 3-O-methyl-D-chiro-inositol+manganese chloride (MnCl₂), D-chiro-inositol+manganese chloride (MnCl₂), manganese sulfate [MnSO4], inositol glycan pseudo-disaccharide Mn(2+) chelate containing D-chiro-inositol 2a (as pinitol) and galactosamine, oral manganese, manganese oxides, e.g., MnO₂, MnOAl₂O₃, and Mn₃O₄.

In one embodiment, the zinc or manganese compound of the present invention is an insulin-mimetic.

Preferred sites of interest in the patient include sites in need of cartilage healing and areas adjacent and/or contiguous to these sites. Local administration of a zinc or manganese compound can be carried out by any means known to a person of ordinary skill in the art.

The term “therapeutically effective amount,” as used herein, means an amount at which the administration of an agent is physiologically significant. The administration of an agent is physiologically significant if its presence results in a detectable change in the bone healing process of the patient.

It will be appreciated that actual preferred amounts of a pharmaceutical composition used in a given therapy will vary depending upon the particular form being utilized, the particular compositions formulated, the mode of application, and the particular site of administration, and other such factors that are recognized by those skilled in the art including the attendant physician or veterinarian. Optimal administration rates for a given protocol of administration can be readily determined by those skilled in the art using conventional dosage determination tests.

Dosages of a zinc or manganese compound employable with the present invention may vary depending on the particular use envisioned. The determination of the appropriate dosage or route of administration is well within the skill of an ordinary physician.

For example, when in vivo administration of a zinc or manganese compound is employed, normal dosage amounts may vary from about 10 ng/kg up to about 100 mg/kg of mammal body weight or more per day, preferably about 1 g/kg/day to 10 mg/kg/day, depending upon the route of administration. Guidance as to particular dosages and methods of delivery is provided in the literature; see, for example, U.S. Pat. Nos. 4,657,760; 5,206,344; 5,225,212; 5,871,799; and 6,232,340. It is anticipated that different formulations will be effective for different treatments and different disorders, and that administration intended to treat a specific site or condition, may necessitate delivery in a manner different from that for another site or condition.

The formulations used herein may also contain more than one active compound as necessary for the particular indication being treated, preferably those with complementary activities that do not adversely affect each other. Alternatively. or in addition, the formulation may comprise a cytotoxic agent, cytokine or growth inhibitory agent. Such molecules are present in combinations and amounts that are effective for the intended purpose.

Therapeutic formulations of zinc or manganese compounds in the zinc or manganese delivery systems employable in the methods of the present invention are prepared for storage by mixing the zinc or manganese compound having the desired degree of purity with optional pharmaceutically acceptable carriers, excipients, or stabilizers (Remington's Pharmaceutical Sciences 16th edition, Osol, A. Ed. (1980)). Such therapeutic formulations can be in the form of lyophilized formulations or aqueous solutions. Acceptable biocompatible carriers, excipients, or stabilizers are nontoxic to recipients at the dosages and concentrations employed, and may include buffers, for example, phosphate, citrate, and other organic acids; antioxidants including ascorbic acid and methionine; preservatives (e.g. octadecyldimethylbenzyl ammonium chloride; hexamethonium chloride; benzalkonium chloride, benzethonium chloride; phenol, butyl or benzyl alcohol; alkyl parabens, for example, methyl or propyl paraben; catechol; resorcinol; cyclohexanol; 3-pentanol; and m-cresol); low molecular weight (less than about 10 residues) polypeptides; proteins, for example, serum albumin, gelatin, or immunoglobulins; hydrophilic polymers, for example, polyvinylpyrrolidone; amino acids, for example, glycine, glutamine, asparagine, histidine, arginine, or lysine; monosaccharides, disaccharides, and other carbohydrates including glucose, mannose, dextrins, or hyaluronan; chelating agents, for example, EDTA; sugars, for example, sucrose, mannitol, trehalose or sorbitol; salt-forming counter-ions, for example, sodium; metal complexes (e.g. Zn-protein complexes); and/or non-ionic surfactants, for example, TWEEN™, PLURONICS™ or polyethylene glycol (PEG).

In order for the formulations to be used for in vivo administration, they must be sterile. The formulation may be readily rendered sterile by filtration through sterile filtration membranes, prior to or following lyophilization and reconstitution. The therapeutic formulations herein preferably are placed into a container having a sterile access port, for example, an intravenous solution bag or vial having a stopper pierceable by a hypodermic injection needle.

The formulations used herein may also contain more than one active compound as necessary for the particular indication being treated, preferably those with complementary activities that do not adversely affect each other. Alternatively, or in addition, the formulation may comprise a cytotoxic agent, cytokine or growth inhibitory agent. Such molecules are present in combinations and amounts that are effective for the intended purpose.

The zinc or manganese may also be entrapped in microcapsules prepared, for example by coacervation techniques or by interfacial polymerization, for example, hydroxy-methylcellulose or gelatin-microcapsules and poly-(methylmethacrylate) microcapsules, respectively. Such preparations can be administered in colloidal drug delivery systems (for example, liposomes, albumin microspheres, microemulsions, nano-particles and nanocapsules) or in macroemulsions. Such techniques are disclosed in Remington's Pharmaceutical Sciences, 16th Edition (or newer), Osol A. ed. (1980).

Optionally, the zinc or manganese agent in the zinc or manganese delivery systems includes a porous calcium phosphate, non-porous calcium phosphate, hydroxy-apatite, tricalcium phosphate, tetracalcium phosphate, calcium sulfate, calcium minerals obtained from natural bone, inorganic bone, organic bone, or a combination thereof.

Where sustained-release or extended-release administration of zinc or manganese in the zinc or manganese delivery systems is desired, microencapsulation is contemplated. Microencapsulation of recombinant proteins for sustained release has been successfully performed with human growth hormone (rhGH), interferon-α, -β, -γ (rhIFN-α, -β, -γ), interleukin-2, and MN rgp120. Johnson et al., Nat. Med. 2: 795-799 (1996); Yasuda, Biomed. Ther. 27: 1221-1223 (1993); Hora et al., Bio/Technology 8: 755-758 (1990); Cleland, “Design and Production of Single Immunization Vaccines Using Polylactide Polyglycolide Microsphere Systems” in Vaccine Design: The Subunit and Adjuvant Approach, Powell and Newman, eds., (Plenum Press: New York, 1995), pp. 439-462; WO 97/03692, WO 96/40072, WO 96/07399 and U.S. Pat. No. 5,654,010.

Suitable examples of sustained-release preparations include semipermeable matrices of solid hydrophobic polymers containing the zinc or manganese in the zinc or manganese delivery systems, which matrices are in the form of shaped articles, e.g. films, or microcapsules. Examples of sustained-release matrices include one or more polyanhydrides (e.g., U.S. Pat. Nos. 4,891,225; 4,767,628), polyesters, for example, polyglycolides, polylactides and polylactide-co-glycolides (e.g., U.S. Pat. No. 3,773,919; U.S. Pat. No. 4,767,628; U.S. Pat. No. 4,530,840; Kulkarni et al., Arch. Surg. 93: 839 (1966)), polyamino acids, for example, polylysine, polymers and copolymers of polyethylene oxide, polyethylene oxide acrylates, polyacrylates, ethylene-vinyl acetates, polyamides, polyurethanes, polyorthoesters, polyacetylnitriles, polyphosphazenes, and polyester hydrogels (for example, poly(2-hydroxyethyl-methacrylate), or poly(vinylalcohol)), cellulose, acyl substituted cellulose acetates, non-degradable polyurethanes, polystyrenes, polyvinyl chloride, polyvinyl fluoride, poly(vinylimidazole), chlorosulphonated polyolefins, polyethylene oxide, copolymers of L-glutamic acid and .gamma.-ethyl-L-glutamate, non-degradable ethylene-vinyl acetate, degradable lactic acid-glycolic acid copolymers, for example, the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release for over 100 days, certain hydrogels release proteins for shorter time periods. Additional non-biodegradable polymers which may be employed are polyethylene, polyvinyl pyrrolidone, ethylene vinylacetate, polyethylene glycol, cellulose acetate butyrate and cellulose acetate propionate.

Alternatively, sustained-release formulations may be composed of degradable biological materials, for example, bioerodible fatty acids (e.g., palimitic acid, steric acid, oleic acid, and the like). Biodegradable polymers are attractive drug formulations because of their biocompatibility, high responsibility for specific degradation, and ease of incorporating the active drug into the biological matrix. For example, hyaluronic acid (HA) may be crosslinked and used as a swellable polymeric delivery vehicle for biological materials. U.S. Pat. No. 4,957,744; Valle et al., Polym. Mater. Sci. Eng. 62: 731-735 (1991). HA polymer grafted with polyethylene glycol has also been prepared as an improved delivery matrix which reduced both undesired drug leakage and the denaturing associated with long term storage at physiological conditions. Kazuteru. M., J. Controlled Release 59:77-86 (1999). Additional biodegradable polymers which may be used are poly(caprolactone), polyanhydrides, polyamino acids, polyorthoesters, polycyanoacrylates, poly(phosphazines), poly(phosphodiesters), polyesteramides, polydioxanones, polyacetals, polyketals, polycarbonates, polyorthocarbonates, degradable and nontoxic polyurethanes, polyhydroxylbutyrates, polyhydroxyvalerates, polyalkylene oxalates, polyalkylene succinates, poly(malic acid), chitin, and chitosan.

Alternatively, biodegradable hydrogels may be used as controlled-release materials for the zinc or manganese compounds in the zinc or manganese delivery systems. Through the appropriate choice of macromers, membranes can be produced with a range of permeability, pore sizes and degradation rates suitable for different types of zinc or manganese compounds in the zinc or manganese delivery systems.

Alternatively, sustained-release delivery systems for zinc or manganese in the zinc or manganese delivery systems can be composed of dispersions. Dispersions may further be classified as either suspensions or emulsions. In the context of delivery vehicles for a zinc or manganese compound, suspensions are a mixture of very small solid particles which are dispersed (more or less uniformly) in a liquid medium. The solid particles of a suspension can range in size from a few nanometers to hundreds of microns, and include microspheres, microcapsules and nanospheres. Emulsions, on the other hand, are a mixture of two or more immiscible liquids held in suspension by small quantities of emulsifiers. Emulsifiers form an interfacial film between the immiscible liquids and are also known as surfactants or detergents. Emulsion formulations can be both oil in water (o/w) wherein water is in a continuous phase while the oil or fat is dispersed, as well as water in oil (w/o), wherein the oil is in a continuous phase while the water is dispersed. One example of a suitable sustained-release formulation is disclosed in WO 97/25563. Additionally, emulsions for use with a zinc or manganese compound in the present invention include multiple emulsions, microemulsions, microdroplets and liposomes. Micro-droplets are unilamellar phospholipid vesicles that consist of a spherical lipid layer with an oil phase inside. E.g., U.S. Pat. No. 4,622,219 and U.S. Pat. No. 4,725,442. Liposomes are phospholipid vesicles prepared by mixing water-insoluble polar lipids with an aqueous solution.

Alternatively, the sustained-release formulations of zinc or manganese in the zinc or manganese delivery systems may be developed using poly-lactic-coglycolic acid (PLGA), a polymer exhibiting a strong degree of biocompatibility and a wide range of biodegradable properties. The degradation products of PLGA, lactic and glycolic acids, are cleared quickly from the human body. Moreover, the degradability of this polymer can be adjusted from months to years depending on its molecular weight and composition. For further information see Lewis, “Controlled Release of Bioactive Agents from Lactide/Glycolide polymer,” in Biogradable Polymers as Drug Delivery Systems M. Chasin and R. Langeer, editors (Marcel Dekker: New York, 1990), pp. 1-41.

The route of administration of “local zinc” or “local manganese” via a “delivery system” is in accordance with known methods, e.g. via immediate-release, controlled-release, sustained-release, and extended-release means. Preferred modes of administration for the zinc or manganese delivery system include injection directly into afflicted site and areas adjacent and/or contiguous to these site or surgical implantation of the zinc or manganese delivery system directly into afflicted sites and area adjacent and/or contiguous to these sites. This type of system may allow temporal control of release as well as location of release as stated above.

As an illustrated example, zinc or manganese may be continuously administered locally to a site via a delivery pump. In one embodiment, the pump is worn externally (in a pocket or on the belt) and attached to the body with a long, thin, and flexible plastic tubing that has a needle or soft cannula (thin plastic tube), and the cannula or needle is inserted and then left in place beneath the skin. The needle or cannula and tubing can be changed, for example, every 48 to 72 hours. The pump would store the zinc or manganese in a cartridge and release it based on the optimal delivery rate. Optionally, the pump is programmed to give a small dose of a drug continuously through the day and night, which in certain circumstances may be preferred.

When an implantable device coated by a composite surface coating comprising a zinc or manganese compound is used, the coating can be formed by any methods known in the relevant art, for example, without limitation, those disclosed in Petrova, R. and Suwattananont, N., J. Electr. Mat., 34(5):8 (2005)). For example, suitable methods include chemical vapor deposition (CVD), physical vapor deposition (PVD), thermochemical treatment, oxidation, and plasma spraying (Fischer, R. C., Met. Progr. (1986); Habig, K. H., Tribol. Int., 22:65 (1989)). A suitable coating of the present invention may also comprise combinations of multiple, preferably two or three, layers obtained by forming first boron diffusion coating followed by CVD (Zakhariev, Z., et al., Surf. Coating Technol., 31:265 (1987)). Thermochemical treatment techniques have been well investigated and used widely in the industry. This is a method by which nonmetals or metals are penetrated by thermodiffusion followed by chemical reaction into the surface. By thermochemical treatment, the surface layer changes its composition, structure, and properties.

Other suitable coating techniques may include, but are not limited to, carburizing, nitriding, carbonitriding, chromizing, and aluminizing. Among these coating techniques, boronizing, being a thermochemical process, is used to produce hard and wear-resistant surfaces. As a person of ordinary skill in the art would understand, different coating techniques may be used to make the zinc- or manganese-based coatings and coated devices of the present invention in order to have desired properties suitable for specific purposes.

### EXAMPLES

### Example 1

### Use of Zinc Compounds for Cartilage Repair

### Materials and Methods

### The BB Wistar Rat Model

### Animal Source and Origin

Diabetic Resistance (DR) BB Wistar rats used in the study were obtained from a breeding colony at UMDNJ-New Jersey Medical School (NJMS). The rats were housed under controlled environmental conditions and fed ad libitum. All research protocols were approved by the Institutional Animal Care and Use Committee at University of Medicine and Dentistry of New Jersey—New Jersey Medical School.

### Diabetic Resistant BB Wistar Rats

A total of 24 DR BB Wistar rats were utilized in the study. Due to unstable fixation during mechanical testing, three samples were removed. Another sample was removed due to complications associated with a post-operative infection. The remaining 17 animals were used for mechanical testing and were distributed between the control saline (n=6), 0.1 mg/kg zinc chloride (n=2), 1.0 mg/kg zinc chloride (n=3), 3.0 mg/kg zinc chloride (n=3), 6.0 mg/kg zinc chloride (n=4) and 10.0 mg/kg zinc chloride (n=3) groups.

### Closed Femoral Fracture Model

Surgery was performed in DR animals between ages 93 and 99 days using a closed mid-diaphyseal fracture model, on the right femur as described previously.

General anesthesia was administrated by intraperitoneal (IP) injection of ketamine (60 mg/kg) and xylazine (8 mg/kg). The right leg of each rat was shaved and the incision site was cleansed with Betadine and 70% alcohol. An approximately 1 cm medial, parapatellar skin incision was made over the patella. The patella was dislocated laterally and the interchondylar notch of the distal femur was exposed. An entry hole was made with an 18 gauge needle and the femur was reamed with the 18 gauge needle. A Kirschner wire (316LVM stainless steel, 0.04 inch diameter, Small Parts, Inc., Miami Lakes, Fla.) was inserted the length of the medullary canal, and drilled through the trochanter of the femur. The kirschner wire was cut flush with the femoral condyles. After irrigation, the wound was closed with 4-0 vicryl resorbable suture. A closed midshaft fracture was then created unilaterally with the use of a three-point bending fracture machine. X-rays were taken to determine whether the fracture was of acceptable configuration. An appropriate fracture is an approximately mid-diaphyseal, low energy, transverse fracture (FIG. 1). The rats were allowed to ambulate freely immediately post-fracture. This closed fracture model is commonly used to evaluate the efficacy of osseous wound healing devices and drugs.

### Local Zinc Delivery

Zinc Chloride [(ZnCl₂), Sigma Aldrich, St. Louis, Mo.] mixed with a buffer was injected into the intramedullary canal prior to fracture. The buffer consisted of sodium acetate, sodium chloride methyl hydroxybenzoate, and zinc chloride. Doses of 1.0 mg/kg and 3.0 mg/kg zinc chloride were tested and administered at a volume of 0.1 mL.

### Mechanical Testing

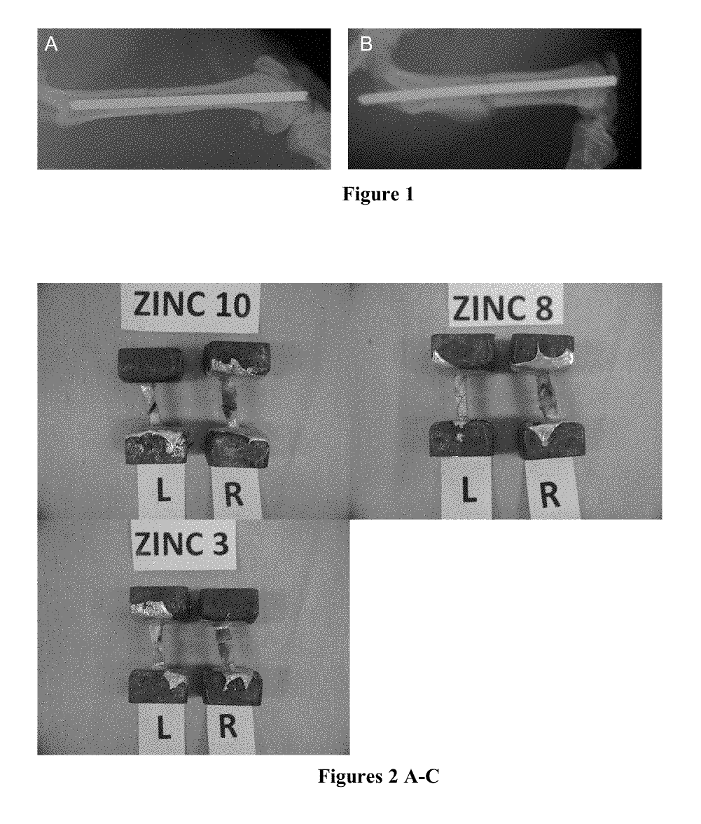

Fractured and contralateral femora were resected at three and four weeks post-fracture. Femora were cleaned of soft tissue and the intramedullary rod was removed. Samples were wrapped in saline (0.9% NaCl) soaked gauze and stored at −20° C. Prior to testing, all femora were removed from the freezer and allowed to thaw to room temperature for three to four hours. The proximal and distal ends of the fractured and contralateral femora were embedded in ¾ inch square nuts with Field's Metal, leaving an approximate gauge length of 18 mm (FIG. 2). After measuring callus, gauge length and femur dimensions, torsional testing was conducted using a servohydraulics machine (MTS Systems Corp., Eden Prairie, Minn.) with a 20 Nmm reaction torque cell (Interface, Scottsdale, Ariz.) and tested to failure at a rate of 2.0 deg/sec. The maximum torque to failure and angle to failure were determined from the force to angular displacement data.

Maximum torque to failure, maximum torsional rigidity, shear modulus, and maximum shear stress were calculated through standard equations (Ekeland, A., et al., Acta Orthop. Scand, 1981, 52(6):605-13; Engesaeter, L. B., et al., Acta Orthop. Scand., 1978, 49(6):512-8). Maximum torque to failure and maximum torsional rigidity are considered extrinsic properties while shear modulus and maximum shear stress are considered intrinsic properties. Maximum torque to failure was defined as the point where an increase in angular displacement failed to produce any further increase in torque. Maximum torsional rigidity is a function of the maximum torque to failure, gauge length (distance of the exposed femur between the embedded proximal and distal end) and angular displacement. Maximum shear stress is a function of the maximum torque to failure, maximum radius within the mid-diaphyseal region and the polar moment of inertia. The polar moment of inertia was calculated by modeling the femur as a hollow ellipse. Engesaeter et al. (1978) demonstrated that the calculated polar moment of inertia using the hollow ellipse model differed from the measured polar moment of inertia by only two percent (Engesaeter, L. B., et al., Acta Orthop. Scand., 1978, 49(6):512-8).

In order to compare the biomechanical parameters between different treatment groups, the data was normalized by dividing each fractured femur value by its corresponding intact, contralateral femur value (FIG. 2). Normalization was used to minimize biological variability due to differences in age and weight among rats.

In addition to the biomechanical parameters determined through torsional testing, the mode of failure can also provide substantial information. The mode of torsional failure as determined by gross inspection provided an indication as to the extent of healing. A spiral failure in the mid-diaphyseal region indicated a complete union while a transverse failure through the fracture site indicated a nonunion. A combination spiral/transverse failure indicated a partial union (FIG. 2).

### Data and Statistical Analysis

Analysis of variance (ANOVA) was performed followed by Holm-Sidak post-hoc tests to determine differences between the treated ZnCl₂ groups with a group size larger than two. A Student's t-test was performed to identify differences between the two treated groups in the ZnCl₂ study (SigmaStat 3.0, SPSS Inc., Chicago, Ill.). A P value less than 0.05 was considered statistically significant.

### General Description of Animal Surgery

A closed mid-diaphyseal fracture surgery was performed on the right femur of each rat as described previously. (Beam, H. A., et al., J. Orthop. Res. 2002, 20(6):1210-1216; Gandhi, A., et al., Bone 2006, 38(4):540-546.) General anesthesia was administered by intraperitoneal injection of ketamine (60 mg/kg) and xylazine (8 mg/kg). A closed, midshaft fracture was then created using a three-point bending fracture instrument (BBC Specialty Automotive, Linden N.J.) and confirmed with X-rays immediately post-fracture.

### Preparation of ZnCl₂ Solution

Zinc chloride (ZnCl₂), Sigma Aldrich, St. Louis, Mo., mixed with sterile water at various doses with or without a calcium sulfate carrier, were injected into the intramedullary canal prior to fracture. Doses of ZnCl₂ were not based on each animal's body weight, but on a lower theoretically tolerable dose for a 290-gram BB Wistar rat, which would not elicit heavy metal poisoning or behavioral changes. This weight is over 50 grams lower than the average weight of non-diabetic BB Wistar rats at an age of approximately 90 days (the age of investigation in this study). A 0.1 ml volume of the ZnCl₂ solution was administered locally via a single injection into the marrow space for each dose examined.

Preparation of ZnCl₂/CaSO₄ Formulation

To prepare the ZnCl₂/CaSO₄ mixture, CaSO₄ (2 g) were placed in glass vials. The vials were placed in an autoclave and sterilized at for two hours in a dry cycle. CaSO₄ powder (0.8 g) was mixed with 400 μl of saline or 400 μl of ZnCl₂ solution (1.0 mg/kg) for one minute at room temperature. The mixture was packed into the barrel of a 1 cc sterile syringe and pushed down into the open orifice of the syringe barrel by insertion of the syringe plunger. After attaching an 18-gauge sterile needle to the syringe barrel, 0.1 ml volume of the mixture was directly injected into the rat femoral canal (non-diabetic BB Wistar rat) prior to Kirschner wire insertion and fracture.

### Microradiographic Evaluation

Serial microradiographs were obtained from all animals every two weeks after surgery. Under the same anesthesia as described above, the rats were positioned prone and lateral and anteroposterior (AP) radiographs of their femurs were obtained. Radiographs were taken using a Packard Faxitron (MX 20—Radiographic Inspection System) and Kodak MinR-2000 mammography film. Exposures were for 30 seconds at 55 kVp. Magnified radiographs were obtained of resected femurs. Qualitative analysis was performed on all radiographic sample at four weeks post-fracture. Two independent observers individually scored radiographs based on bridging of the lateral and AP femoral orientations. Treatment group averages were computed to estimate healing at 4 weeks post-fracture. The analysis was conducted in a blinded fashion using a validated, five-point radiographic scoring system, 0=no evident bony bridging, 1=bony bridging of one cortex, 2=bony bridging of two cortices, 3=bony bridging of three cortices, and 4=bony bridging of all four cortices. (See Bergenstock, M. W., et al., J. Orthop. Trauma 2005, 19(10):717-723.)

### Torsional Mechanical Testing

Torsional testing was conducted at four weeks using a servohydraulics machine (MTS Sys. Corp., Eden Prairie, Minn.) with a 20 Nm reaction torque cell (Interface, Scottsdale, Ariz.). Femurs were tested to failure at a rate of 2.0 deg/sec at four and six week time points. The peak torque, torsional rigidity, effective bulk modulus, and the effective maximum shear stress (a) were determined with standard equations that model each femur as a hollow ellipse. (Ekeland, A., et al., Acta Orthop. Scand. 1981, 52(6):605-613; Engesaeter, L. B., et al., Acta Orthop. Scand. 1978, 49(6):512-518). In order to compare the biomechanical parameters between different groups, the data was normalized by dividing each fractured femur value by its corresponding intact, contralateral femur value. Torsional mechanical testing is limited by differences in gauge length during bone potting in Field's metal. Placement and dimension of fracture gap can contribute to standard deviations. Finally, this test is limited because it relies on a mathematical model that assumes the femur is a hollow ellipse, as opposed to the natural architecture of femoral bone. (Levenston, M. E., et al., J. Bone Miner. Res. 1994, 9(9):1459-1465.)

### Early-Stage Healing Analysis by Histomorphometry

The fractured femora were resected at seven days post-fracture, decalcified, dehydrated, embedded in paraffin, and sectioned using standard histological techniques. Sections were stained with Masson's Trichrome (Accustain™ Trichrome Staining kit, Sigma Diagnostics, St. Louis, Mo.) for histological observation using an Olympus BH2-RFCA microscope (Olympus Optical Co., Ltd., Shinjuku-ku, Tokyo, Japan). Digital images were collected using a Nikon DXM1200F digital camera (Nikon, Tokyo, Japan). Cartilage, new bone, and total callus area were measured from the digital images using Image-Pro Plus software (version 5, Media Cybernetics, Inc., Silver Spring, Md.). Total cartilage and new bone area were normalized to total callus area and expressed as the percent area. Two independent reviewers were used to minimize inconsistencies.

### Late-Stage Healing Analysis by Histomorphometry

To examine the effects of VAC at later stages of fracture healing, femora were resected from animals in the groups described above at day 21, embedded and sectioned using standard histological techniques. This includes dehydration, soaking in Xylenes, and finally pre-embedding in a layer of Polymethylmethacrylate (PMMA). After embedding in pure PMMA and allowed to solidify in a hot water bath, slides were sectioned from the PMMA blocks, polished, and stained with a combination of Stevenel's blue and Van Gieson picro-fuchsin (SVG). Histological images of fracture calluses were obtained using an Olympus SZX12 upright microscope (Olympus Optical Co, LTD, Japan) connected via a CCD camera (Optronics, Goleta, Calif.) to a personal computer and analyzed with the Bioquant software package (Biometrics, Inc, Nashville, Tenn.). Parameters that were compared include a) callus area, b) percent calcified tissue area, and c) percent cartilage area. Limitations of this procedure include production of slides with high thicknesses, due to the difficulties associated with sectioning PMMA. This limits the number of possible sections that may be cut for staining in addition to analysis of cellular morphology, due to overlapping layers of cells.

### General Health of Animals

The age of the BB Wistar rats at the time of fracture surgery varied between 75 and 137 days. However, animals amongst treatment groups were age and sex matched for each experiment. The percent weight change following surgery to the day of sacrifice was similar amongst treatment groups.

### Results

### General Health

In this experiment, the rats were 93-117 days old at time of fracture. No significant difference in percent weight gain was found between treatment groups from time of fracture until euthanization (Table 2). Blood glucose levels were higher in the zinc chloride treated rats, but the blood glucose values were within the normal range for all treatment groups (Table 2).

TABLE 2
General health of non-DM BB Wistar rats: local zinc
(ZnCl₂) delivery without a carrier (Mechanical Testing)

| | Blood Glucose (mg/dl)* | % Weight |
|---|---|---|
| | 12 Hours Post-Surgery | gain |
| Saline Control | 81.7 ± 4.3 ^{a} | 3.5 ± 2.3 |
| (n = 6) | | |
| 0.1 mg/kg ZnCl₂ | 87.0 ± 7.1 ^{a} | 15.3 ± 11.5 |
| (n = 2) | | |
| 1.0 mg/kg ZnCl₂ | 99.3 ± 3.1 ^{b} | 11.0 ± 9.4 |
| (n = 3) | | |
| 3.0 mg/kg ZnCl₂ | 105.0 ± 4.4 ^{b} | 6.9 ± 11.7 |
| (n = 3) | | |
| 6.0 mg/kg ZnCl₂ | 88.0 ± 4.3 ^{a} | 4.6 ± 2.3 |
| (n = 4) | | |
| 10.0 mg/kg ZnCl₂ | 87.7 ± 8.5 ^{a} | 4.2 ± 2.0 |
| (n = 3) | | |

The data represents average values ± standard deviation
^{a} represents values significantly less than the 3.0 mg/kg ZnCl₂ group; p < 0.05
^{b} represents values significantly less than the saline group; p < 0.05

### Microradiographic Evaluation

At four weeks post-fracture, femurs from rats treated with ZnCl₂ had significantly higher radiograph scores than control femurs (Table 3).

### Mechanical Testing Results

### Local ZnCl₂ (No Carrier)

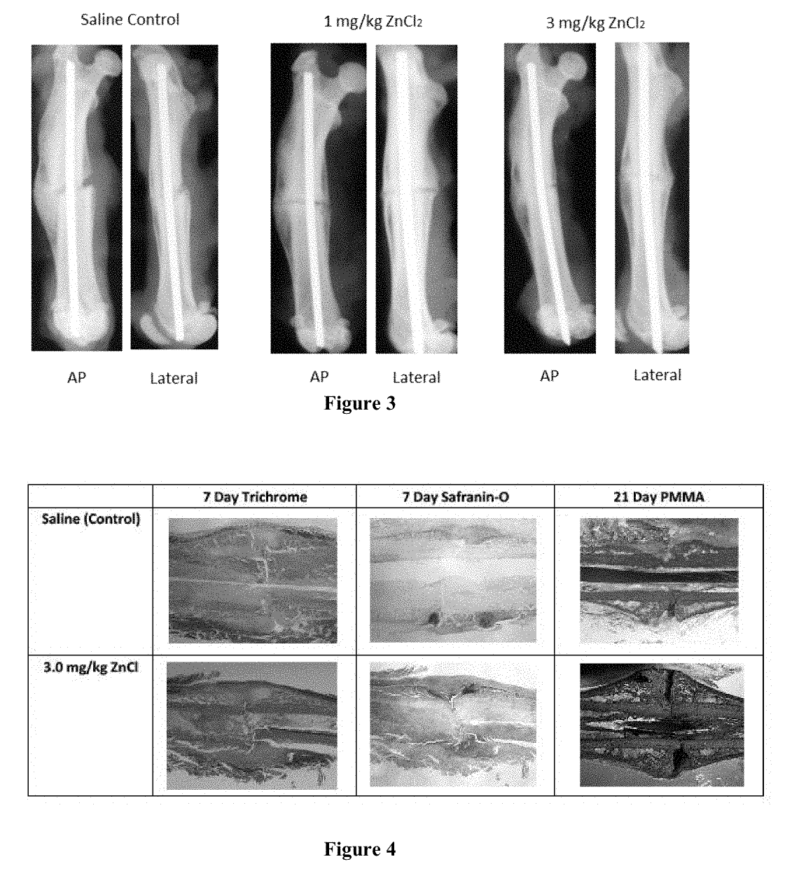

The effect of local zinc therapy on healing of femur fractures was measured by torsional mechanical testing. At four weeks post-fracture, rats treated with local ZnCl₂ displayed improved mechanical properties of the fractured femora compared to the untreated group. Radiographs taken at 4 weeks post-fracture support this finding (FIG. 3). Table 3 represents the radiograph scoring values at two different dosages.

TABLE 3
Radiographic scoring evaluation

| | 4 Weeks Post-Fracture |
|---|---|
| | (# of cortices bridged) |
| Saline Control | 1.2 ± 0.75 |
| (n = 6) | (n = 6) |
| 1.0 mg/kg ZnCl₂ | 3.0 ± 0.6* |
| (n = 3) | (n = 3) |
| 3.0 mg/kg ZnCl₂ | 3.3 ± 0.6* |
| (n = 3) | (n = 3) |

The data represents average values ± standard deviation
*Represent values statistically higher than control, p < 0.05

Table 4 summarizes the results of the mechanical testing of the bone for fractured bone, following four weeks of healing. The effective shear stress was 1.6× and 2.2× higher at four weeks post-fracture for the healing femurs from the ZnCl₂-treated animals, at dosages of 1.0 mg/kg and 3.0 mg/kg respectively. When normalized to their intact, contralateral femurs, the percent maximum torque to failure, percent torsional rigidity, and percent effective shear modulus, of the fractured femora were 2.0×, 3.8×, and 8.0× higher, respectively, at the dosage of 3 mg/kg ZnCl₂ compared to the control group (p<0.05).

The effect of local zinc therapy on healing of femur fractures in normal (non-diabetic) rats was measured by torsional mechanical testing. At 4 weeks post-fracture, fractured femurs from the rats treated with zinc chloride had greater mechanical properties than the fractured femurs from the control group. For the 10 mg/kg ZnCl₂ group, the maximum torsional rigidity was significantly greater than the untreated group (Table 4). When the mechanical parameters of the fractured femora were normalized to the intact, contralateral femora, percent maximum torque to failure (saline group vs. 3 mg/kg ZnCl₂ group p<0.05), torsional rigidity (saline group vs. 3 mg/kg ZnCl₂ group p<0.05), and shear modulus (Saline group vs. 3 mg/kg ZnCL₂ group p<0.05, Saline group vs. 10 mg/kg ZnCL₂ group p<0.05) were significantly greater in the local zinc treated groups when compared to the saline group (Table 4).

Healing was assessed by radiographic examination and quantified by mechanical testing. Local ZnCl₂ treatment improved radiographic appearance and significantly increased the mechanical strength of fractured femurs. At four weeks post-fracture, the average percent maximum torque to failure of the fractured femora for 3.0 mg/kg ZnCl₂ was significantly (2.04 times) greater (82.0% of contralateral vs. 27.0%), compared to the untreated saline group. Percent maximum torsional rigidity values for 3.0 mg/kg ZnCl₂ was significantly (3.85 times) greater (97.0% of contralateral vs. 20.0%), compared to the untreated saline group. Percent shear modulus values for both low (3.0 mg/kg ZnCl₂) and high (10.0 mg/kg ZnCl₂) doses were significantly greater, with high dose 8.8 times greater (36.0% of contralateral vs. 4.0%), and low dose 9.0 times greater (39.0% of contralateral vs. 4.0%) compared to the untreated saline group. The data indicate that local ZnCl₂ treatment enhanced bone regeneration during fracture healing and indicates that zinc and potentially similar metals can be used as therapeutically as osteogenic drugs.

TABLE 4
Four weeks post-fracture mechanical
testing with local zinc (ZnCl₂)
Fractured Femur Values

| | Maximum | Maximum | Effective | Effective |
|---|---|---|---|---|
| | Torque to | Torsional | Shear | Shear |
| | Failure | Rigidity | Modulus | Stress |
| | (Nmm) | (Nmm²/rad) | (MPa) | (MPa) |
| Saline | 161 ± 48 | 9.9 × 10³ ± | 2.6 × 10² ± | 17 ± 4 |
| Control | | 4.7 × 10³ | 1.1 × 10² | |
| (n = 6) | | | | |
| 0.1 mg/kg | 252 ± 13 | 2.1 × 10⁴ ± | 1.7 × 10³ ± | 61 ± 14 |
| ZnCl₂ (n = 2) | | 4.2 × 10³ | 3.3 × 10² | |
| 1.0 mg/kg | 281 ± 86 | 2.2 × 10⁴ ± | 9.7 × 10² ± | 44 ± 15 |
| ZnCl₂ (n = 3) | | 2.7 × 10³ | 3.6 × 10² | |
| 3.0 mg/kg | 369 ± 74 | 3.1 × 10⁴ ± | 1.3 × 10³ ± | 55 ± 21* |
| ZnCl₂ (n = 3) | | 1.1 × 10⁴ | 6.4 × 10² | |
| 6.0 mg/kg | 276 ± 190 | 2.9 × 10⁴ ± | 1.1 × 10³ ± | 32 ± 25* |
| ZnCl₂ (n = 4) | | 1.6 × 10⁴ | 7.5 × 10² | |
| 10.0 mg/kg | 254 ± 36 | 3.6 × 10⁴ ± | 3.0 × 10³ ± | 62 ± 30 |
| ZnCl₂ (n = 3) | | 2.5 × 10⁴ | 1.9 × 10³* | |

Fractured Femur Values Normalized to the Contralateral (Intact) Femur

| | Percent | Percent | Percent | |
|---|---|---|---|---|
| | Maximum | Maximum | Effective | Percent |
| | Torque to | Torsional | Shear | Effective |
| | Failure | Rigidity | Modulus | Shear Stress |
| Saline | 27 ± 18 | 20 ± 10 | 4 ± 2 | 10 ± 5 |
| Control | | | | |
| (n = 6) | | | | |
| 0.1 mg/kg | 57 ± 12 | 87 ± 14 | 34 ± 4 | 33 ± 14 |
| ZnCl₂ (n = 2) | | | | |
| 1.0 mg/kg | 65 ± 29 | 55 ± 14 | 32 ± 15 | 18 ± 8 |
| ZnCl₂ (n = 3) | | | | |
| 3.0 mg/kg | 82 ± 25* | 97 ± 55* | 36 ± 10* | 27 ± 17 |
| ZnCl₂ (n = 3) | | | | |
| 6.0 mg/kg | 38 ± 20 | 62 ± 35 | 18 ± 12 | 15 ± 10 |
| ZnCl₂ (n = 4) | | | | |
| 10.0 mg/kg | 41 ± 8 | 73 ± 44 | 39 ± 23* | 27 ± 11 |
| ZnCl₂ (n = 3) | | | | |

The data represents average values ± standard deviation
*Represents values statistically higher than saline control, p < 0.05 versus saline control.
One way ANOVA between 6 groups (all pairwise) with a Holm-Sidak post-hoc analysis

### Histomorphometry of Zinc Chloride Treated Fractures

The results of histomorphometry of zinc chloride treated fractures after 7, 10, and 21 days are listed in Table 5 and illustrated in FIG. 4.

TABLE 5
Histomorphometry of zinc chloride-treated fractures

| | |
|---|---|
| % Bone | % Cartilage |

7 Day

| | | |
|---|---|---|
| Saline Control | 8.08 ± 2.45 | 3.00 ± 1.7 |
| (n = 5) | | |
| 3.0 mg/kg | 18.92 ± 5.97* | 4.64 ± 3.41 |
| (n = 7) | | |

10 Day

| | | |
|---|---|---|
| Saline Control | 17.90 ± 5.20 | 16.3 ± 2.8 |
| (n = 5) | | |
| 3.0 mg/kg | 21.31 ± 5.40 | 12.79 ± 3.02 |
| (n = 7) | | |

21 Day

| | | |
|---|---|---|
| Saline Control | 25.00 ± 6.10 | 6.1 ± 3.2 |
| (n = 6) | | |
| 3.0 mg/kg | 24.47 ± 3.53 | 11.57 ± 5.53 |
| (n = 7) | | |

Local ZnCl₂/CaSO₄ Formulations

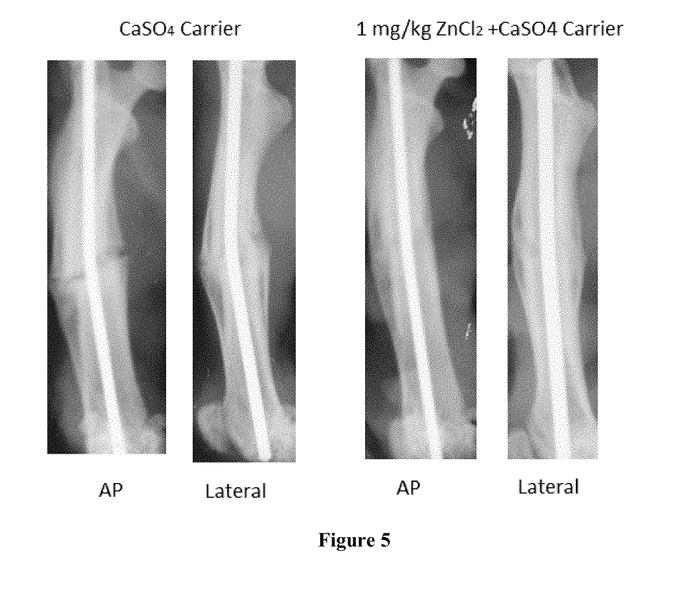

We repeated the above experiment with formulations of ZnCl₂/CaSO₄ applied to the fracture site. Radiographs taken at four weeks post-fracture support this finding (FIG. 5) shows significant bone formation.

TABLE 6
Four weeks post-fracture mechanical testing with formulation of zinc
chloride (ZnCl₂) with CaSO₄ carrier applied to the fracture site.
Fractured Femur Values

| | Maximum | Maximum | Effective | Effective |
|---|---|---|---|---|
| | Torque to | Torsional | Shear | Shear |
| | Failure | Rigidity | Modulus | Stress |
| | (Nmm) | (Nmm²/rad) | (MPa) | (MPa) |
| Saline | 161 ± 48 | 9.9 × 10³ ± | 2.6 × 10² ± | 17 ± 4 |
| Control | | 4.7 × 10³ | 1.1 × 10² | |
| (n = 6) | | | | |
| CaSO₄ | 251 ± 78 | 2.1 × 10⁴ ± | 6.0 × 10² ± | 26 ± 10 |
| Control | | 1.3 × 10⁴ | 3.7 × 10² | |
| (n = 7) | | | | |
| 0.5 mg/kg | 337 ± 175 | 3.0 × 10⁴ ± | 1.1 × 10³ ± | 36 ± 22 |
| ZnCl2 + | | 7.9 × 10³ | 9.4 × 10² | |
| CaSO₄ (n = 4) | | | | |
| 1.0 mg/kg | 396 ± 112* | 3.9 × 10⁴ ± | 1.3 × 10³ ± | 46 ± 16* |
| ZnCl2 + | | 1.4 × 10⁴*,^{#} | 7.1 × 10²* | |
| CaSO₄ (n = 7) | | | | |
| 3.0 mg/kg | 262 ± 126 | 2.1 × 10⁴ ± | 7.0 × 10² ± | 33 ± 19 |
| ZnCl2 + | | 7.8 × 10³ | 3.1 × 10² | |
| CaSO₄ (n = 5) | | | | |

Fractured Femur Values Normalized to the Contralateral (Intact) Femur

| | Percent | Percent | Percent | Percent |
|---|---|---|---|---|
| | Maximum | maximum | Effective | Effective |
| | Torque to | Torsional | Shear | Shear |
| | Failure | Rigidity | Modulus | Stress |
| Saline | 27 ± 18 | 20 ± 10 | 4 ± 2 | 10 ± 5 |
| Control | | | | |
| (n = 6) | | | | |
| CaSO₄ | 48 ± 21 | 55 ± 35 | 11 ± 7 | 16 ± 7 |
| Control | | | | |
| (n = 7) | | | | |
| 0.5 mg/kg | 56 ± 31 | 63 ± 20 | 17 ± 19 | 19 ± 12 |
| ZnCl2 + | | | | |
| CaSO₄ (n = 4) | | | | |
| 1.0 mg/kg | 75 ± 18* | 79 ± 32* | 18 ± 10 | 27 ± 8* |
| ZnCl2 + | | | | |
| CaSO₄ (n = 7) | | | | |
| 3.0 mg/kg | 45 ± 22 | 52 ± 22 | 14 ± 8 | 20 ± 14 |
| ZnCl2 + | | | | |
| CaSO₄ (n = 5) | | | | |

The data represents average values ± standard deviation
*Represents values statistically higher than saline control, p < 0.05 versus saline control.
^{#}Represents values statistically higher than CaSO4 control, p < 0.05 versus CaSO4 control.
One-way ANOVA between 5 groups with Holm-Sidak post-hoc analysis

Table 6 summarizes the results of the mechanical testing of the bone for fractured bone, following four weeks of healing using the formulation. The effective shear stress was 2.7× and 1.7× higher at four weeks post-fracture for the healing femurs from the ZnCl₂/CaSO₄ treated animals, at dosages of 1.0 mg/kg compared to saline and CaSO₄ control, respectively. When normalized to their intact, contralateral femurs, the percent maximum torque to failure, percent torsional rigidity, and percent effective shear modulus, of the fractured femora were 2.8×, 4.0×, and 4.5× higher, respectively, at the dosage of 1 mg/kg ZnCl₂ CaSO₄ compared to the saline control group (p<0.05).

Comparison of Use of ZnCl₂ with Existing Therapy (BMP2)

As an insulin-mimetic adjunct, zinc compounds can be used to accelerate bone regeneration by stimulating insulin signaling at the fracture site. ZnCl₂ treatment applied directly to the fracture site significantly increased the mechanical parameters of the bone in treated animals after four weeks, compared to controls. It accelerated fracture-healing process (fracture healing resolved in four to five weeks, instead of average eight to ten weeks in standard rat femur fracture model).

Other healing adjuncts currently approved for FDA use in the United States include Bone Morphogenic Proteins (BMP's) and Exogen/Pulsed Electromagnetic Fields (PEMF). However, BMPs may be associated with shortcomings such as causing ectopic bone growth and having high cost per application; and Exogen/PEMF therapy has shown only limited proven usefulness in fracture healing and needs for patient compliance for daily use.

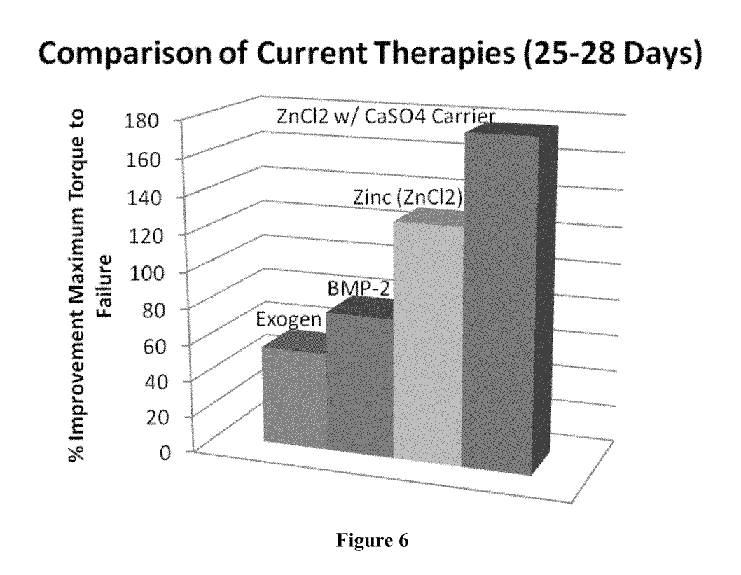

The chart in FIG. 6 compares the use of ZnCl₂ (alone or in combination with CaSO₄) with the currently approved products (BMP-2 and Exogen) for fracture healing. Each of these studies examined the effectiveness of a therapeutic adjunct on femur fracture healing by measuring the maximum torque to failure at the four week time point. Specifically the following were compared to their respective untreated control group:

(1) a single intramedullary dose (1 mg/kg) of ZnCl2 with the calcium sulfate (CaSO₄) vehicle (purple); (2) a single intramedullary dose (3 mg/kg) of ZnCl2 without a vehicle (green); (3) BMP-2 study used a single percutaneous dose of BMP-2 (80 mg) with buffer vehicle (red) (see Einhorn, T. A., et al., J. Bone Joint Surg. Am. 2003, 85-A(8):1425-1435); and (4) Exogen study used daily exposure periods of ultrasound treatment (20 min/day). The average value (duration of 25 days) is shown in blue (see Azuma, Y., et al., J. Bone Miner. Res. 2001, 16(4):671-680.

As graphically shown, use of single application of insulin-mimetic like zinc chloride results in significantly increased improvement of torque to failure and other mechanical properties of the fracture callus, compared to the existing gold standard of LIPUS and BMP2, using torsional mechanical testing of rat femur fracture model of Bonnarrens and Einhorn.

In summary, we have found that acute, local ZnCl₂ treatment (either alone or as a formulation with a carrier), administered immediately prior to an induced fracture, promoted healing in non-diabetic rats. At the four week time point, mechanical parameters of the healed bone were substantially higher than that of the control group. This is consistent with our earlier findings of insulin's ability to promote bone growth when applied to the fracture site. This is also consistent with our finding that insulin mimetic compounds such as vanadyl acetylacetonate (VAC) accelerate fracture healing much like insulin. Though also an insulin mimetic, unlike VAC, ZnCl₂ is a compound commonly used in many commercial medical products and hence potential regulatory barriers are minimal. This suggests that insulin mimetics applied locally to the fracture may be used therapeutically as a fracture-healing adjunct, and local ZnCl₂ treatment is a cost-effective fracture-healing adjunct and has potential for other possible orthopedic applications.

The above preliminary data indicate that local treatment with an insulin-mimetic such as zinc is an effective method to enhance bone regeneration. Mechanical parameters and radiography revealed that bone bridged at four weeks after fracture in the zinc-treated rats as compared to saline treated controls. Spiral fractures that occurred during mechanical testing support the radiographic observations and suggest that local ZnCl₂ application at the dosages tested may accelerate fracture healing, compared to untreated controls. These data support additional testing of ZnCl₂ as a therapeutic agent to accelerate or enhance bone regeneration.

### Example 2

### Use of Manganese Compounds for Fracture Healing

### Material and Methods

### Rat Model

The animal model used for this study is the Diabetes Resistant (DR) BB Wistar Rat. It will be obtained from a breeding colony at UMDNJ-New Jersey Medical School (NJMS) which is maintained under controlled environmental conditions and fed ad libitum.

The BB Wistar colony was established from diabetic-prone BB Wistar rats originally obtained from BioBreeding (Toronto, Canada). Similar to human type I diabetes, spontaneously diabetic BB Wistar rats display marked hyperglycemia, glycosuria and weight loss within a day of onset, associated with decreased plasma insulin after undergoing selective and complete destruction of pancreatic β-cells. If left untreated, diabetic BB Wistar rats would become ketoacidic within several days, resulting in death. Genetic analysis of the BB-Wistar rat shows the development of diabetes is strongly related to the presence of the iddm4 diabetogenic susceptibility locus on chromosome 4 as well as at least four other loci related to further susceptibility and the development of lymphopenia (Martin, A. M., et al., Diabetes 1999, 48(11):2138-44).

The DR-BB Wistar rat colony was also originally purchased from BioBreeding and has been established as an effective control group for studies involving the diabetic BB Wistar rat. Under controlled environmental conditions, DR-BB Wistar rats would never develop spontaneous type I diabetes, are non-lymphopenic, and are immunocompetent. It has since been used in our lab as a model of a “normal” rat model. The choice was made to utilize the DR-BB Wistar rat, rather than purchase commercially available rats for our studies, because of the ability to expand the colony by breeding at any time as necessary for different protocols, as well our familiarity with the rat over years of its utilization in similar protocols. The consistent use of the BB Wistar and the DR-BB Wistar rat models allow for an increase in reliability when comparing data between our various protocols.

### General Health of Animals

The age of the BB Wistar rats at the time of fracture surgery varied between 95 and 137 days. However, animals amongst treatment groups were age and sex matched for each experiment. The percent weight change following surgery to the day of sacrifice was similar amongst treatment groups.

### Surgical Technique

Surgery will be performed to produce a closed mid-diaphyseal fracture model in the right femur. General anesthesia will be administered prior to surgery by intraperitoneal (IP) injection of ketamine (60 mg/kg) and xylazine (8 mg/kg). The right leg of each rat is shaved and the incision site is prepared with Betadine and 70% alcohol. A one centimeter medial, parapatellar skin incision is made, followed by a smaller longitudinal incision through the quadriceps muscle, just proximal to the quadriceps tendon. The patella is dislocated laterally and the intercondylar notch of the distal femur is exposed. An entry hole is made with an 18-gauge needle and the femoral intramedullary canal is subsequently reamed. For experimental groups, 0.1 mL of MnCl2 solution (of different dosage) is injected into the medullary canal of the femur. For control groups, 0.1 mL of saline is injected. A Kirschner wire (316LVM stainless steel, 0.04 inch diameter, Small Parts, Inc., Miami Lakes, Fla.) is inserted into the intramedullary canal. The Kirschner wire is cut flush with the femoral condyles. After irrigation, the wound is closed with 4-0 vicryl resorbable sutures. A closed midshaft fracture is then created unilaterally with the use of a three-point bending fracture machine. X-rays are taken to determine whether the fracture is of acceptable configuration. Only transverse, mid-diaphyseal fractures are accepted. The rats are allowed to ambulate freely immediately post-fracture.

### Post Surgery Procedures

X-rays are taken at two-week intervals to the day of euthanasia. After euthanasia x-rays are taken as well. To take x-rays, animals will be given a half dose of anesthesia. All groups will be monitored closely for four days after surgery for infection, and the ability to ambulate freely.

### Torsional Mechanical Testing

Torsional testing was conducted at 4 weeks post-fracture, using a servohydraulics machine (MTS Sys. Corp., Eden Prairie, Minn.) with a 20 Nm reaction torque cell (Interface, Scottsdale, Ariz.). Femurs were tested to failure at a rate of 2.0 deg/sec at four weeks post-fracture. The peak torque, torsional rigidity, effective bulk modulus, and the effective maximum shear stress (a) were determined with standard equations that model each femur as a hollow ellipse (Ekeland, A., et al., Acta Orthop. Scand. 1981, 52(6):605-613; Engesaeter, L. B., et al., Acta Orthop. Scand. 1978, 49(6):512-518). In order to compare the biomechanical parameters between different groups, the data was normalized by dividing each fractured femur value by its corresponding intact, contralateral femur value. Torsional mechanical testing is limited by differences in gauge length during bone potting in Field's metal. Placement and dimension of fracture gap can contribute to standard deviations. Finally, this test is limited because it relies on a mathematical model that assumes the femur is a hollow ellipse, as opposed to the natural architecture of femoral bone (Levenston, M. E., et al., J. Bone Miner. Res. 1994, 9(9):1459-1465).

### Early-Stage Healing Analysis by Histomorphometry

The fractured femora were resected at seven and ten days post-fracture, decalcified, dehydrated, embedded in paraffin, and sectioned using standard histological techniques. Sections were stained with Masson's Trichrome (Accustain™ Trichrome Staining kit, Sigma Diagnostics, St. Louis, Mo.) for histological observation using an Olympus BH2-RFCA microscope (Olympus Optical Co., Ltd., Shinjuku-ku, Tokyo, Japan). Digital images were collected using a Nikon DXM1200F digital camera (Nikon, Tokyo, Japan). Cartilage, new bone, and total callus area were measured from the digital images using Image-Pro Plus software (version 5, Media Cybernetics, Inc., Silver Spring, Md.). Total cartilage and new bone area were normalized to total callus area and expressed as the percent area. Two independent reviewers were used to minimize inconsistencies.

### Data and Statistical Analysis

Analysis of variance (ANOVA) was performed followed by Holm-Sidak post-hoc tests to determine differences between the treated MnCl₂ groups with a group size larger than two. A Student's t-test was performed to identify differences between the two treated groups in the MnCl₂ study (SigmaStat 3.0, SPSS Inc., Chicago, Ill.). A p value less than 0.05 was considered statistically significant.

### Results

### Mechanical Testing

### Local MnCl₂ No Carrier

The effect of local MnCl₂ therapy on healing of femur fractures was measured by torsional mechanical testing. At four weeks post-fracture, rats treated with MnCl₂ displayed improved mechanical properties of the fractured femora compared to the saline control group. The maximum torque to failure was significantly increased compared to the saline control group (p<0.05: 0.125 mg/kg MnCl₂, p<0.05: 0.25 mg/kg MnCl₂, p<0.05: 0.3 mg/kg MnCl₂) (Table 7). When the mechanical parameters of the fractured femora were normalized to the intact, contralateral femora, percent torsional rigidity was significantly greater in the local MnCl₂ treated groups when compared to the saline control group (p<0.05: 0.125 mg/kg MnCl₂, p<0.05: 0.25 mg/kg MnCl₂) (Table 7).

TABLE 7
Four weeks post-fracture mechanical testing
with local manganese chloride (MnCl²)
Fractured Femur Values

| | Maximum | Maximum | Effective | Effective |
|---|---|---|---|---|
| | Torque to | Torsional | Shear | Shear |
| | Failure | Rigidity | Modulus | Stress |
| | (Nmm) | (Nmm²/rad) | (MPa) | (MPa) |
| Saline Control | 161 ± 48 | 9.9 × 10³ ± | 2.6 × 10² ± | 17 ± 4 |
| (n = 6) | | 4.7 × 10³ | 1.1 × 10² | |
| 0.083 mg/kg | 272 ± 39 | 2.6 × 10⁴ ± | 8.7 × 10² ± | 30 ± 8 |
| MnCl₂ (n = 5) | | 1.2 × 10⁴ | 4.9 × 10² | |
| 0.125 mg/kg | 351 ± 59* | 4.2 × 10⁴ ± | 6.4 × 10² ± | 21 ± 6 |
| MnCl₂ (n = 4) | | 1.1 × 10⁴ | 8.8 × 10¹ | |
| 0.25 mg/kg | 344 ± 84* | 3.4 × 10⁴ ± | 8.1 × 10² ± | 32 ± 11 |
| MnCl₂ (n = 4) | | 1.6 × 10⁴ | 5.0 × 10² | |
| 0.30 mg/kg | 323 ± 135* | 3.0 × 10⁴ ± | 7.6 × 10² ± | 27 ± 23 |
| MnCl₂ (n = 6) | | 2.6 × 10⁴ | 9.2 × 10² | |
| 0.50 mg/kg | 230 ± 83 | 2.9 × 10⁴ ± | 6.2 × 10² ± | 19 ± 9 |
| MnCl₂ (n = 6) | | 1.2 × 10⁴ | 3.5 × 10² | |

Fractured Femur Values Normalized to the Contralateral (Intact) Femur

| | Percent | Percent | Percent | Percent |
|---|---|---|---|---|
| | Maximum | maximum | Effective | Effective |
| | Torque to | Torsional | Shear | Shear |
| | Failure | Rigidity | Modulus | Stress |
| Saline Control | 27 ± 18 | 20 ± 10 | 4 ± 2 | 10 ± 5 |
| (n = 6) | | | | |
| 0.083 mg/kg | 42 ± 5 | 56 ± 30 | 8 ± 7 | 8 ± 4 |
| MnCl₂ (n = 5) | | | | |
| 0.125 mg/kg | 54 ± 5 | 103 ± 40* | 16 ± 11 | 14 ± 5 |
| MnCl₂ (n = 4) | | | | |
| 0.25 mg/kg | 55 ± 19 | 80 ± 34* | 14 ± 9 | 16 ± 6 |
| MnCl₂ (n = 4) | | | | |
| 0.30 mg/kg | 50 ± 22 | 50 ± 37 | 10 ± 12 | 16 ± 12 |
| MnCl₂ (n = 6) | | | | |
| 0.50 mg/kg | 38 ± 15 | 61 ± 16 | 17 ± 13 | 14 ± 7 |
| MnCl₂ (n = 6) | | | | |

The data represents average values ± standard deviation
*Represents values statistically higher than saline control, p < 0.05 versus saline control.

### Radiographic Analysis

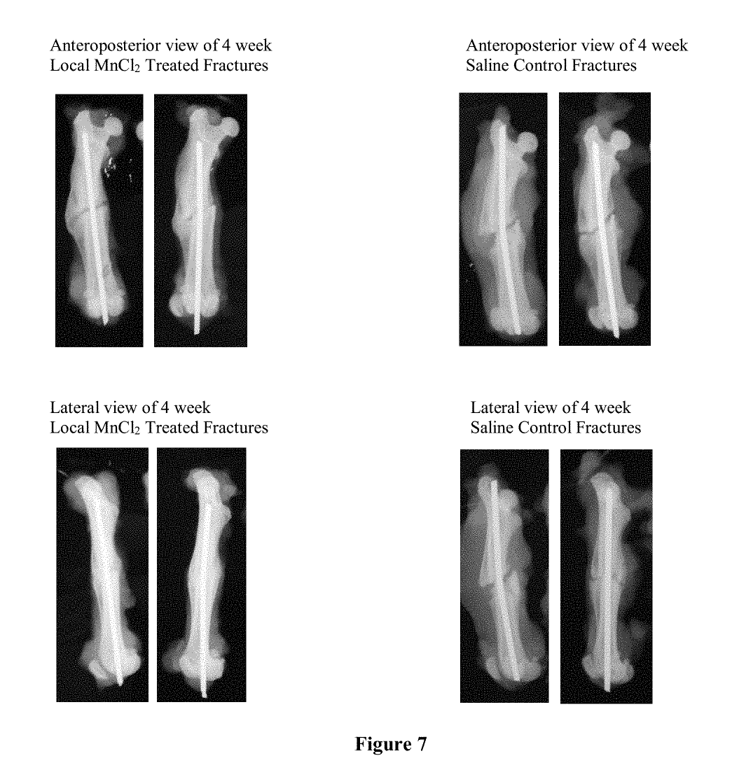

Radiographs taken at four weeks post-fracture support these mechanical testing results (FIG. 7). At four weeks, the fractures treated with 0.25 mg/kg dosage of MnCl₂ displayed increased mineralized tissue than saline controls. Additionally, analysis of radiographs showed the MnCl₂ group demonstrated union at the subperiosteal bony area and at the callus, whereas saline control radiographs had no evidence of union.

### Histomorphometric Analysis

In animals treated with MnCl2, histomorphometric analysis revealed a statistically lower (p<0.05) percent cartilage in 0.3 mg/kg MnCl₂ treated femora, compared to controls at seven days (Table 8). At ten days, percent mineralized tissue in 0.3 mg/kg MnCl₂ treated femora were significantly increased (p<0.05: 0.3 mg/kg MnCl₂) compared to saline controls (Table 8).

TABLE 8
Histology: comparison of manganese chloride with saline control

| | 7 days post fracture | | 10 days post fracture | |
|---|---|---|---|---|
| Group | % cartilage | % new bone | % cartilage | % new bone |
| Saline | 6.116 ± 2.51 | 15.668 ± 2.93 | 9.542 ± 1.02 | 14.011 ± 1.29 |
| 0.3 mg/kg | 2.859 ± 1.09 ^{#} | 15.604 ± 2.39 | 11.051 ± 3.05 | 18.866 ± 2.28 * |

* Represents values statistically higher than saline control, p < 0.001
^{#} Represents values statistically lower than saline control, p < 0.05

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and script of the invention, and all such variations are intended to be included within the scope of the following claims.

All references cited hereby are incorporated by reference in their entirety.

What is claimed is:

1. A method for repairing an injury of cartilage tissue in a patient in need thereof, comprising locally administering a therapeutically effective amount of zinc or manganese compound to said patient.

2. The method of claim 1, wherein said zinc or manganese compound is a zinc compound.

3. The method of claim 2, wherein said zinc compound is an inorganic zinc compound selected from the group consisting of zinc chloride, zinc sulfate, zinc phosphate, zinc carbonate, and zinc nitrate.

4. The method of claim 2, wherein said zinc compound is an organic acid zinc salt selected from the group consisting of zinc acetate, zinc formate, zinc propionate, zinc gluconate, bis(maltolato)zinc, zinc acexamate, zinc aspartate, bis(maltolato)zinc(II) [Zn(ma)2], bis(2-hydroxypyridine-N-oxido)zinc(II) [Zn(hpo)2], bis(allixinato)Zn(II) [Zn(alx)2], bis(6-methylpicolinato)Zn(II) [Zn(6mpa)2], bis(aspirinato)zinc(II), bis(pyrrole-2-carboxylato)zinc [Zn(pc)2], bis(alpha-furonic acidato)zinc [Zn(fa)2], bis(thiophene-2-carboxylato)zinc [Zn(tc)2], bis(thiophene-2-acetato)zinc [Zn(ta)2], (N-acetyl-L-cysteinato)Zn(II) [Zn(nac)], zinc(II)/poly(γ-glutamic acid) [Zn(γ-pga)], bis(pyrrolidine-N-dithiocarbamate)zinc(II) [Zn(pdc)₂], zinc(II) L-lactate [Zn(lac)₂], zinc(II) D-(2)-quinic acid [Zn(qui)₂], bis(1,6-dimethyl-3-hydroxy-5-methoxy-2-pentyl-1,4-dihydropyridine-4-thionato)zinc(II) [Zn(tanm)2], β-alanyl-L-histidinato zinc(II) (AHZ).

5. The method of claim 1, wherein said zinc or manganese compound is a manganese compound.

6. The method of claim 5, wherein said manganese compound is manganese chloride (MnCl₂).

7. The method according to claim 1, wherein said cartilage injury is selected from the group consisting of traumatic cartilaginous injuries, osteochondral lesions, osteochondral fracture, osteochondritis dissecans, chondromalacia, avascular necrosis, chemical induced cartilage damage, and genetic cartilage deficiency.

8. The method according to claim 1, wherein said cartilage is an articular cartilage.

9. The method according to claim 1, wherein the method is used in combination with arthroscopic debridement, marrow stimulating techniques, autologous chondrocyte transfers, and autologous chondrocyte implantation, and allografts.

10. The method according to claim 1, wherein the method is used in conjunction with administration of a cytototoxic agent, cytokine or growth inhibitory agent.

11. The method according to claim 1, wherein the method is used in conjunction with an allograft, autograft or orthopedic biocomposite.

12. The method according to claim 1, wherein said patient is a mammalian animal.

13. The method according to claim 1, wherein said patient is a human, a horse, or a dog.

14. The method according to claim 1, wherein said patient is a non-diabetic human.

15. (canceled)

16. A method for repairing an injury of a cartilage in a patient in need thereof, comprising applying to the site of said injury an implantable device comprising a zinc or manganese compound.

17. The method of claim 16, wherein said zinc or manganese compound is a zinc compound.

18. The method of claim 17, wherein said zinc compound is an inorganic zinc compound selected from the group consisting of zinc chloride, zinc sulfate, zinc phosphate, zinc carbonate, and zinc nitrate.

19. The method of claim 17, wherein said zinc compound is an organic acid zinc salt selected from the group consisting of zinc acetate, zinc formate, zinc propionate, zinc gluconate, bis(maltolato)zinc, zinc acexamate, zinc aspartate, bis(maltolato)zinc(II) [Zn(ma)2], bis(2-hydroxypyridine-N-oxido)zinc(II) [Zn(hpo)2], bis(allixinato)Zn(II) [Zn(alx)2], bis(6-methylpicolinato)Zn(II) [Zn(6mpa)2], bis(aspirinato)zinc(II), bis(pyrrole-2-carboxylato)zinc [Zn(pc)2], bis(alpha-furonic acidato)zinc [Zn(fa)2], bis(thiophene-2-carboxylato)zinc [Zn(tc)2], bis(thiophene-2-acetato)zinc [Zn(ta)2], (N-acetyl-L-cysteinato)Zn(II) [Zn(nac)], zinc(II)/poly(γ-glutamic acid) [Zn(γ-pga)], bis(pyrrolidine-N-dithiocarbamate)zinc(II) [Zn(pdc)₂], zinc(II) L-lactate [Zn(lac)₂], zinc(II) D-(2)-quinic acid [Zn(qui)₂], bis(1,6-dimethyl-3-hydroxy-5-methoxy-2-pentyl-1,4-dihydropyridine-4-thionato)zinc(II) [Zn(tanm)2], β-alanyl-L-histidinato zinc(II) (AHZ).

20. The method of claim 16, wherein said zinc or manganese compound is a manganese compound.

21. The method of claim 20, wherein said manganese compound is manganese chloride (MnCl₂).

22. The method according to claim 16, wherein said cartilage injury is selected from the group consisting of traumatic cartilaginous injuries, osteochondral lesions, osteochondral fracture, osteochondritis dissecans, chondromalacia, avascular necrosis, chemical induced cartilage damage, and genetic cartilage deficiency.

23. The method according to claim 16, wherein said cartilage injury is that of an articular cartilage.

24. The method according to claim 16, wherein the method is used in conjunction with arthroscopic debridement, marrow stimulating techniques, autologous chondrocyte transfers, and autologous chondrocyte implantation, and allografts.

25. The method according to claim 16, wherein the method is used in conjunction with administration of a cytototoxic agent, cytokine or growth inhibitory agent.

26. The method according to claim 16, wherein the method is used in conjunction with an allograft, autograft or orthopedic biocomposite.

27. The method according to claim 16, wherein said patient is a mammalian animal.

28. The method according to claim 16, wherein said patient is a human, a horse, or a dog.

29. The method according to claim 16, wherein said patient is a non-diabetic human.

30. (canceled)

31. An implantable device for implant in a cartilage to repair an injury of the cartilage, said implantable device comprising a zinc or manganese compound.

32. The implantable device of claim 31, wherein said zinc or manganese compound is a zinc compound.

33. The implantable device of claim 32, wherein said zinc compound is an inorganic zinc compound selected from the group consisting of zinc chloride, zinc sulfate, zinc phosphate, zinc carbonate, and zinc nitrate.

34. The implantable device of claim 32, wherein said zinc compound is an organic acid zinc salt selected from the group consisting of zinc acetate, zinc formate, zinc propionate, zinc gluconate, bis(maltolato)zinc, zinc acexamate, zinc aspartate, bis(maltolato)zinc(II) [Zn(ma)2], bis(2-hydroxypyridine-N-oxido)zinc(II) [Zn(hpo)2], bis(allixinato)Zn(II) [Zn(alx)2], bis(6-methylpicolinato)Zn(II) [Zn(6mpa)2], bis(aspirinato)zinc(II), bis(pyrrole-2-carboxylato)zinc [Zn(pc)2], bis(alpha-furonic acidato)zinc [Zn(fa)2], bis(thiophene-2-carboxylato)zinc [Zn(tc)2], bis(thiophene-2-acetato)zinc [Zn(ta)2], (N-acetyl-L-cysteinato)Zn(II) [Zn(nac)], zinc(II)/poly(γ-glutamic acid) [Zn(γ-pga)], bis(pyrrolidine-N-dithiocarbamate)zinc(II) [Zn(pdc)₂], zinc(II) L-lactate [Zn(lac)₂], zinc(II) D-(2)-quinic acid [Zn(qui)₂], bis(1,6-dimethyl-3-hydroxy-5-methoxy-2-pentyl-1,4-dihydropyridine-4-thionato)zinc(II) [Zn(tanm)2], β-alanyl-L-histidinato zinc(II) (AHZ).

35. The implantable device of claim 31, wherein said zinc or manganese compound is a manganese compound.

36. The implantable device of claim 35, wherein said manganese compound is manganese chloride (MnCl₂).

37. The implantable device according to claim 31, coated by a composite surface coating comprising a zinc or manganese compound.

38. The implantable device of claim 37, wherein said zinc or manganese compound is zinc.

39. The implantable device of claim 38, wherein said zinc compound is an inorganic zinc compound selected from the group consisting of zinc chloride, zinc sulfate, zinc phosphate, zinc carbonate, and zinc nitrate.

40. The implantable device of claim 38, wherein said zinc compound is an organic acid zinc salt selected from the group consisting of zinc acetate, zinc formate, zinc propionate, zinc gluconate, bis(maltolato)zinc, zinc acexamate, zinc aspartate, bis(maltolato)zinc(II) [Zn(ma)2], bis(2-hydroxypyridine-N-oxido)zinc(II) [Zn(hpo)2], bis(allixinato)Zn(II) [Zn(alx)2], bis(6-methylpicolinato)Zn(II) [Zn(6mpa)2], bis(aspirinato)zinc(II), bis(pyrrole-2-carboxylato)zinc [Zn(pc)2], bis(alpha-furonic acidato)zinc [Zn(fa)2], bis(thiophene-2-carboxylato)zinc [Zn(tc)2], bis(thiophene-2-acetato)zinc [Zn(ta)2], (N-acetyl-L-cysteinato)Zn(II) [Zn(nac)], zinc(II)/poly(γ-glutamic acid) [Zn(γ-pga)], bis(pyrrolidine-N-dithiocarbamate)zinc(II) [Zn(pdc)₂], zinc(II) L-lactate [Zn(lac)₂], zinc(II) D-(2)-quinic acid [Zn(qui)₂], bis(1,6-dimethyl-3-hydroxy-5-methoxy-2-pentyl-1,4-dihydropyridine-4-thionato)zinc(II) [Zn(tanm)2], β-alanyl-L-histidinato zinc(II) (AHZ).

41. The implantable device of claim 37, wherein said zinc or manganese compound is a manganese compound.

42. The implantable device of claim 41, wherein said manganese compound is manganese chloride (MnCl₂).

43. (canceled)

44. (canceled)

45. (canceled)

46. (canceled)

47. (canceled)

48. (canceled)
